(19) United States
(12) Patent Application Publication
(10) Pub. No.: US 2005/0074802 A1
(43) Pub. Date: Apr. 7, 2005

(54) Nucleotide sequences which code for the MetD gene

(75) Inventors: Daniel Rey, Leopoldshoehe (DE); Christian Rueckert, Guetersloh (DE); Joern Kalinowski, Bielefeld (DE); Alfred Puehler, Bielefeld (DE); Brigitte Bathe, Salzkotten (DE); Klaus Huthmacher, Gelnhausen (DE); Walter Pfefferle, Halle (Westf.) (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/936,597

(22) Filed: Sep. 9, 2004

Related U.S. Application Data
(62) Division of application No. 10/156,856, filed on May 30, 2002.

(30) Foreign Application Priority Data
May 30, 2001 (DE) 101 26 164.0

(51) Int. Cl. C12Q 1/68; C07H 21/04; C12N 9/10; C12N 9/88; C12N 15/74

(52) U.S. Cl. 435/6; 435/69.1; 435/193; 435/252.3; 435/471; 536/23.2

(57) ABSTRACT

The invention relates to polynucleotides comprising polynucleotide sequences corresponding to the metD gene and parts thereof that encode polypeptide sequences and parts thereof possessing varying degrees of MetD transcription regulator activity, methods for preparation of L-amino acids, and methods of screening and amplifying polynucleotides encoding polypeptide sequences which comprise varying degrees of MetD transcription regulator activity. Further, the invention relates to animal food additives based on fermentation liquor and containing L-methionine, and to the preparation of such additive.

### BACKGROUND OF INVENTION

1. Field of the Invention

The invention relates to polynucleotides comprising polynucleotide sequences corresponding to the metD gene and parts thereof that encode polypeptide sequences and parts thereof possessing varying degrees of MetD transcription regulator activity, methods for preparation of L-amino acids, and methods of screening and amplifying polynucleotides encoding polypeptide sequences which comprise varying degrees of MetD transcription regulator activity. Further, the invention relates to animal food additives based on fermentation liquor and containing L-methionine, and to the preparation of such additive.

2. Discussion of the Background

L-Amino acids, in particular L-methionine, are used in human medicine and in the pharmaceuticals industry, in the foodstuffs industry, and, very particularly, in animal nutrition.

It is known that amino acids are prepared by fermentation from strains of Coryneform bacteria, in particular Corynebacterium glutamicum. Because of their great importance, work is constantly being undertaken to improve the preparation processes. Improvements to the process can relate to fermentation measures, such as, stirring and supply of oxygen, or the composition of the nutrient media, such as, the sugar concentration during the fermentation, or the working up to the product form by, for example, ion exchange chromatography, or the intrinsic output properties of the microorganism itself.

Methods of mutagenesis, selection, and mutant selection are used to improve the output properties of these microorganisms. Strains which are resistant to antimetabolites or are auxotrophic for metabolites of regulatory importance and which produce amino acids are obtained in this manner.

Methods of the recombinant DNA technique have also been employed for some years for creating Coryneform bacterium strains, which produce L-amino acid by amplifying individual amino acid biosynthesis genes and investigating the effect on the amino acid production. During the time preceding the present invention, however, it was not known that attenuated expression of the metD gene would improve L-amino acid production yields.

### SUMMARY OF THE INVENTION

An object of the present invention is to provide novel measures for improved preparation of L-amino acids or amino acids where these amino acids include L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, including their salts (such as methionine hydrochloride or methionine sulfate).

Another object of the present invention is a novel process for improving fermentative preparation of the L-amino acids, L-Methionine in particular. This process includes enhanced bacteria, preferably from Coryneform bacteria, which express attenuated amounts of MetD transcription regulator, which is encoded by the metD gene. Another object of the present invention is to provide a fermentation broth comprising an L-methionine-producing microorganism.

Another object of the present invention is to provide an animal food additive, which may be made by a process comprising the steps of:
a) culturing an L-methionine-producing microorganism in a fermentation medium;
b) concentrating the L-methionine-containing fermentation broth;
c) removing an amount of from 0 to 100 wt. % of the biomass formed during the fermentation; and
d) drying of the fermentation broth obtained according to a) and/or b) to obtain the animal feedstuffs additive in the desired powder or granule form.

Another object of the present invention is to provide a method of preparing an animal food additive comprising further steps of:
e) adding at least one organic substances, including L-methionine and/or D-methionine and/or the racemic mixture D,L-methionine, to the products obtained according to a), b) and/or c);
f) adding auxiliary substances chosen from the group consisting of silicas, silicates, stearates, grits and bran to the substances obtained according to a) to d) for stabilization and to increase the storability; or
g) converting the substances obtained according to a) to e) into a form stable to the animal stomach, in particular rumen, by coating with film-forming agents.

Another object of the present invention is to provide such a bacterium, preferably from Coryneform bacteria, which expresses attenuated metD gene products.

Another object of the present invention is to provide such a bacterium, preferably from Coryneform bacteria, which expresses attenuated MetD transcription regulator activity.

Another object of the present invention is to provide a polynucleotide sequence encoding a polypeptide sequence with MetD transcription regulator activity. One embodiment of such a sequence is the polynucleotide sequence of SEQ ID NO. 1.

Another object of the present invention is a method of making MetD transcription regulator or a polypeptide having MetD transcription regulator activity. One embodiment of such a sequence is the polypeptide sequence of SEQ ID NO. 2.

Other objects of the present invention include methods of detecting polynucleotides that are homologous to SEQ ID NO: 1 or those polynucleotides encoding polypeptides that have having MetD transcription regulator activity, methods of making such polynucleotides encoding such polypeptides, and methods of making such polypeptides.

The above descriptions highlight certain aspects and embodiments of the present invention. Additional objects, aspects, and embodiments of the present invention follow in the detailed description of the present invention considered together with the Figures.

### BRIEF DESCRIPTION OF THE FIGURES

FIG. 1: Map of the plasmid pK18mobsacBmetD del

### DETAILED DESCRIPTION OF THE INVENTION

Unless specifically defined, all technical and scientific terms used herein have the same meaning as commonly understood by a skilled artisan of molecular biology.

“Isolated” refers to a material, i.e. a polynucleotide separated out of its natural environment.

“Polynucleotide” in general relates to polyribonucleotides and polydeoxyribonucleotides, it being possible for these to be non-modified RNA or DNA or modified RNA or DNA.

The term “attenuation” in this connection describes the reduction or elimination of the intracellular activity of one or more enzymes (proteins) in a microorganism which are coded by the corresponding DNA, for example, by using a weak promoter or using a gene or allele which codes for a corresponding enzyme with a low activity or inactivates the corresponding gene or enzyme (protein), and optionally combining these measures. By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

“Polypeptides” are understood as meaning peptides or proteins, which comprise two or more amino acids, bonded via peptide bonds.

The term “enhancement” in this connection describes an increase in the intracellular activity of one or more enzymes (proteins) in a microorganism which are coded by the corresponding DNA, for example, by increasing the number of copies of the gene or genes, using a potent promoter or using a gene or allele which codes for a corresponding enzyme (protein) having a high activity, and optionally combining these measures. By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. Further, the materials, methods, and examples are illustrative only and are not intended to be limiting.

Reference is made to standard textbooks of molecular biology that contain definitions and methods and means for carrying out basic scientific techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and various references cited therein.

The invention provides an isolated polynucleotide from Coryneform bacteria, comprising a polynucleotide sequence, which codes for the metD gene, chosen from the group consisting of
(a) polynucleotide which is identical to the extent of at least 70% to a polynucleotide which codes for a polypeptide, which comprises the amino acid sequence of SEQ ID NO. 2,
(b) polynucleotide which codes for a polypeptide, which comprises an amino acid sequence, which is identical to the extent of at least 70% to the amino acid sequence of SEQ ID NO. 2,
(c) polynucleotide which is complementary to the polynucleotides of a) or b), and
(d) polynucleotide comprising at least 15 successive nucleotides of the polynucleotide sequence of a), b) or c), the polypeptide preferably has the activity of MetD transcription regulator.

“Transcriptional regulators” are defined herein as proteins which are able to increase or decrease the transcription level of specific genes by binding at certain DNA regions.

It has been found that some transcriptional regulators have a specific structure called helix-turn-helix-motif. The invention provides the function of the transcriptional regulator metD as the repression of genes which are involved in L-amino acid biosyntheses in particular the biosynthesis of L-methionine. The attenuation of the transcriptional regulator metD improves the production of L-methionine in Coryneform bacteria.

The invention also provides the above-mentioned polynucleotide, this preferably being a DNA which is capable of replication, comprising:
(i) the nucleotide sequence, shown in SEQ ID NO.1, or
(ii) at least one sequence which corresponds to sequence (i) within the range of the degeneration of the genetic code, or
(iii) at least one sequence which hybridizes with the sequences complementary to sequences (i) or (ii), and optionally
(iv) sense mutations of neutral function in (i).

The invention also provides at least one polynucleotides chosen from the group consisting of:
a) polynucleotides comprising at least 15 successive nucleotides chosen from the nucleotide sequence of SEQ ID No. 1 between positions 1 and 313,
b) polynucleotides comprising at least 15 successive nucleotides chosen from the nucleotide sequence of SEQ ID No. 1 between positions 314 and 1024,
c) polynucleotides comprising at least 15 successive nucleotides chosen from the nucleotide sequence of SEQ ID No. 1 between positions 1025 and 1322.

Further, the invention also provides:
(a) a polynucleotide, in particular DNA, which is capable of replication and comprises the nucleotide sequence as shown in SEQ ID NO.1;
(b) a polynucleotide, which codes for a polypeptide which, comprises the amino acid sequence as shown in SEQ ID NO. 2;
(c) a vector containing parts of the polynucleotide according to the invention, but at least 15 successive nucleotides of the sequence claimed,
(d) Coryneform bacteria in which the metD gene is attenuated, in particular by an insertion or deletion.

The invention also provides polynucleotides with a polynucleotide sequence which comprises the complete metD gene or parts thereof, obtainable by screening by means of hybridization of a corresponding gene library of a Coryneform bacterium with a probe which comprises the sequence of the polynucleotide according to SEQ ID NO.1 or a fragment thereof, and isolation of the polynucleotide sequence mentioned.

The present invention provides polynucleotides which comprise the sequences according to the invention are suitable as hybridization probes for RNA, cDNA and DNA, in order to isolate, in the fill length, nucleic acids or polynucleotides or genes which code for MetD transcription regulator or to isolate those nucleic acids or polynucleotides or genes which have a high similarity with the sequence of the metD gene. They are also suitable for incorporation into so-called “arrays”, “micro arrays” or “DNA chips” in order to detect and to determine the corresponding polynucleotides.

Polynucleotides, which comprise the sequences according to the invention, are furthermore suitable as primers, which code for MetD transcription regulator can be prepared by the polymerase chain reaction (PCR).

Such oligonucleotides which serve as probes or primers comprise at least 25, 26, 27, 28, 29 or 30, preferably at least 20, 21, 22, 23 or 24, very particularly preferably at least 15, 16, 17, 18 or 19 successive nucleotides. Oligonucleotides which have a length of at least 31, 32, 33, 34, 35, 36, 37, 38, 39 or 40 or at least 41, 42, 43, 44, 45, 46, 47, 48, 49 or 50 nucleotides are also suitable. Oligonucleotides with a length of at least 100, 150, 200, 250 or 300 nucleotides are optionally also suitable.

The polynucleotides according to the invention include a polynucleotide according to SEQ ID NO. 1 or a fragment prepared therefrom and also those which are at least 70% to 80%, preferably at least 81% to 85%, particularly preferably at least 86% to 90%, and very particularly preferably at least 91%, 93%, 95%, 97% or 99% identical to the polynucleotide according to SEQ ID NO. 1 or a fragment prepared therefrom.

The polypeptides according to the invention include a polypeptide according to SEQ ID NO. 2, in particular those with the biological activity of MetD transcription regulator, and also those which are at least 70% to 80%, preferably at least 81% to 85%, particularly preferably at least 86% to 90%, and very particularly preferably at least 91%, 93%, 95%, 97% or 99% identical to the polypeptide according to SEQ ID NO. 2 and have the activity mentioned.

The invention furthermore relates to a process for the fermentative preparation of amino acids chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using Coryneform bacteria which, in particular, already produce amino acids and in which the nucleotide sequences which code for the metD gene are attenuated, in particular eliminated or expressed at a low level.

The increase of the protein concentration can be analyzed by 1- and 2-dimensional protein gel electrophoresis followed by optical identification of the protein concentration while using specific computer software. A common method to prepare protein gels using Coryneform bacteria and to identify the proteins is described by Hermann et al. (Electrophoresis, 22: 1712-23 (2001)).

The concentration of the protein can be also analyzed by Western blot hybridization techniques while using specific antibodies (Sambrook et al., Molecular Cloning: A Laboratory Manual. 2nd Ed., Cold Spring Harbor Laboratory Press, Cold Spring Harbor, N.Y., 1989) and subsequent optical evaluation with computer software commonly used for analyzing of protein concentrations (Lohaus und Meyer (1998) Biospektrum 5: 32-39; Lottspeich (1999) Angewandte Chemie 111: 2630-2647). The activity of DNA binding proteins can be measured by DNA band shift assays (also known as gel retardation (Wilson et al. (2001) Journal of Bacteriology 183: 2151-2155). The influence of DNA binding proteins on gene expression can be identified by well described reporter gene assays (Sambrook et al., Molecular cloning: a laboratory manual. 2nd Ed. Cold Spring Harbor Laboratory Press, Cold Spring Harbor, N.Y., 1989).

The microorganisms to which the present invention relates can prepare amino acids from glucose, sucrose, lactose, fructose, maltose, molasses, starch, cellulose or from glycerol and ethanol. They can be representatives of Coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum (C. glutamicum), are in particular the known wild-type strains
Corynebacterium glutamicum ATCC 13032
Corynebacterium aceloglutamicum ATCC 15806
Corynebacterium acetoacidophilum ATCC13870
Corynebacterium melassecola ATCC 17965
Corynebacterium thermoaminogenes FERM BP-1539
Brevibacterium flavum ATCC 14067
Brevibacterium lactofermentum ATCC13869 and
Brevibacterium divaricatum ATCC14020
   or L-amino acid-producing mutants or strains prepared therefrom, as, for example the methionine-producing strain Corynebacterium glutamicum ATCC21608.

Preferably, a bacterial strand with attenuated expression of metD gene products with MetD transcription regulator activity will improve amino acid yields at least 1%.

The inventors have isolated the new metD gene from C. glutamicum, which codes for MetD transcription regulator.

To isolate the metD gene or also other genes of C. glutamicum, a gene library of this microorganism is first set up in Escherichia coli (E. coli). The setting up of gene libraries is described in generally known textbooks and handbooks. The textbook by Winnacker: Gene und Klone, Eine Einführung in die Gentechnologie [Genes and Clones, An Introduction to Genetic Engineering] (Verlag Chemie, Weinheim, Germany, 1990), or the handbook by Sambrook et al.: Molecular Cloning, A Laboratory Manual (Cold Spring Harbor Laboratory Press, 1989) may be mentioned as an example. A well-known gene library is that of the E. coli K-12 strain W3110 set up in λ vectors by Kohara et al. (Cell 50, 495-508 (1987)). Bathe et al. (Molecular and General Genetics, 252: 255-265, 1996) describe a gene library of C. glutamicum ATCC13032, which was set up with the aid of the cosmid vector SuperCos I (Wahl et al., 1987, Proceedings of the National Academy of Sciences USA, 84: 2160-2164) in the E. coli K-12 strain NM554 (Raleigh et al., 1988, Nucleic Acids Research 16: 1563-1575). Bormann et al. (Molecular Microbiology 6 (3), 317-326)) (1992)) in turn describe a gene library of C. glutamicum ATCC13032 using the cosmid pHC79 (Hohn and Collins, 1980, Gene 11, 291-298).

To prepare a gene library of C. glutamicum in E. coli, it is also possible to use plasmids such as pBR322 (Bolivar, 1979, Life Sciences, 25, 807-818) or pUC9 (Vieira et al., 1982, Gene, 19: 259-268). Suitable hosts are, in particular, those E. coli strains which are restriction- and recombination-defective, such as the strain DH5αmcr, which has been described by Grant et al. (Proceedings of the National Academy of Sciences USA, 87 (1990) 4645-4649).

The long DNA fragments cloned with the aid of cosmids or other λ vectors can then in turn be subcloned and subsequently sequenced in the usual vectors which are suitable for DNA sequencing, such as is described e.g. by Sanger et al. (Proceedings of the National Academy of Sciences of the United States of America, 74: 5463-5467, 1977).

The resulting DNA sequences can then be investigated with known algorithms or sequence analysis programs, such as e.g. that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new DNA sequence of C. glutamicum which codes for the metD gene and which, as SEQ ID NO. 1, is a constituent of the present invention has been found in this manner. The amino acid sequence of the corresponding protein has furthermore been derived from the present DNA sequence by the methods described above. The resulting amino acid sequence of the metD gene product is shown in SEQ ID NO. 2.

Coding DNA sequences, which result from SEQ ID NO. I by the degeneracy of the genetic code, are also a constituent of the invention. In the same way, DNA sequences, which hybridize with SEQ ID NO. 1 or parts of SEQ ID NO. 1, are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. are of neutral function. It is furthermore known that changes on the N and/or C terminus of a protein cannot substantially impair or can even stabilize the function thereof. Information in this context can be found by the expert, inter alia, in Ben-Bassat et al. (Journal of Bacteriology 169: 751-757 (1987)), in O'Regan et al. (Gene 77: 237-251 (1989)), in Sahin-Toth et al. (Protein Sciences 3: 240-247 (1994)), in Hochuli et al. (Bio/Technology 6: 1321-1325 (1988)) and in known textbooks of genetics and molecular biology. Amino acid sequences, which result in a corresponding manner from SEQ ID NO. 2, are also a constituent of the invention.

In the same way, DNA sequences, which hybridize with SEQ ID NO. 1 or parts of SEQ ID NO. 1, are a constituent of the invention. Finally, DNA sequences, which are prepared by the polymerase chain reaction (PCR) using primers, which result from SEQ ID NO. 1, are a constituent of the invention. Such oligonucleotides typically have a length of at least 15 nucleotides.

The skilled artisan will find instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (International Journal of Systematic Bacteriology 41: 255-260 (1991)). The hybridization takes place under stringent conditions, that is to say only hybrids in which the probe and target sequence, i.e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5×SSC buffer at a temperature of approx. 50° C.-68° C., for example, can be employed for the hybridization reaction. Probes can also hybridize here with polynucleotides, which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) a temperature of approx. 50° C.-68° C. being established. It is optionally possible to lower the salt concentration to 0.1×SSC. Polynucleotide fragments which are, for example, at least 70% or at least 80% or at least 90% to 95% identical to the sequence of the probe employed can be isolated by increasing the hybridization temperature stepwise from 50° C. to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

A skilled artisan will find instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

The inventors have shown that Coryneform bacteria produce amino acids in an improved manner after attenuation of the metD gene.

To achieve attenuation, either the expression of the metD gene or the catalytic properties of the enzyme protein can be reduced or eliminated. The two measures can optionally be combined.

The reduction in gene expression can take place by suitable culturing or by genetic modification (mutation) of the signal structures of gene expression. Signal structures of gene expression are, for example, repressor genes, activator genes, operators, promoters, attenuators, ribosome binding sites, the start codon and terminators. The expert can find information on this e.g. in WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Patek et al. (Microbiology 142: 1297 (1996)), Vasicova et al. (Journal of Bacteriology 181: 6188 (1999)) and in known textbooks of genetics and molecular biology, such as e.g. the textbook by Knippers (“Molekulare Genetik [Molecular Genetics]”, 6th edition, Georg Thieme Verlag, Stuttgart, Germany, 1995) or that by Winnacker (“Gene und Klone [Genes and Clones]”, VCH Verlagsgesellschaft, Weinheim, Germany, 1990).

Mutations which lead to a change or reduction in the catalytic properties of enzyme proteins are known from the prior art; examples which may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Mockel (“Die Threonindehydratase aus Corynebacterium glutamicum: Aufhebung der allosterischen Regulation und Struktur des Enzyms [Threonine dehydratase from Corynebacterium glutamicum: Canceling the allosteric regulation and structure of the enzyme]”, Reports from the Jülich Research Center, Jül-2906, ISSN09442952, Jülich, Germany, 1994). Summarizing descriptions can be found in known textbooks of genetics and molecular biology, such as e.g. that by Hagemann (“Allgemeine Genetik [General Genetics]”, Gustav Fischer Verlag, Stuttgart, 1986).

Possible mutations are transitions, transversions, insertions and deletions. These mutations may be referred to as “missense mutations” or “nonsense mutations”, depending on the effect of the amino acid exchange on the enzyme activity. Insertions or deletions of at least one base pair (bp) in a gene lead to frame shift mutations, as a consequence of which incorrect amino acids are incorporated or translation is interrupted prematurely. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik [Molecular Genetics]”, 6th edition, Georg Thieme Verlag, Stuttgart, Germany, 1995), that by Winnacker (“Gene und Klone [Genes and Clones]”, VCH Verlagsgesellschaft, Weinheim, Germany, 1990) or that by Hagemann (“Allgemeine Genetik [General Genetics]”, Gustav Fischer Verlag, Stuttgart, 1986).

A common method of mutating genes of C. glutamicum is the method of “gene disruption” and “gene replacement” described by Schwarzer and Puhler (Bio/Technology 9, 84-87 (1991)).

In the method of gene disruption a central part of the coding region of the gene of interest is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega Corporation, Madison, Wis., USA), pCR2.1-TOPO (Shuman (1994). Journal of Biological Chemistry 269: 32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173: 4510-4516). The plasmid vector, which contains the central part of the coding region of the gene, is then transferred into the desired strain of C. glutamicum by conjugation or transformation. The method of conjugation is described, for example, by Schäfer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” event, the coding region of the gene in question is interrupted by the vector sequence and two incomplete alleles are obtained, one lacking the 3′ end and one lacking the 5′ end. This method has been used, for example, by Fitzpatrick et al. (Applied Microbiology and Biotechnology 42, 575-580 (1994)) to eliminate the recA gene of C. glutamicum.

In the method of “gene replacement”, a mutation, such as e.g. a deletion, insertion or base exchange, is established in vitro in the gene of interest. The allele prepared is in turn cloned in a vector, which is not replicative for C. glutamicum, and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch et al. (Microbiology 144, 915-927 (1998)) to eliminate the pyc gene of C. glutamicum by a deletion.

A deletion, insertion or a base exchange can be incorporated into the metD gene in this manner.

In addition, it may be advantageous for the production of L-amino acids to enhance, in particular over-express, one or more enzymes of the particular biosynthesis pathway, of glycolysis, of anaplerosis, of the citric acid cycle, of the pentose phosphate cycle, of amino acid export and optionally regulatory proteins, in addition to the attenuation of the metD gene.

The use of endogenous genes is in general preferred. The term “endogenous genes” or “endogenous nucleotide sequences” is understood to mean the genes or nucleotide sequences present in the population of a species.

Thus, for example, for the preparation of L-amino acids, in addition to the attenuation of the metD gene at the same time one or more of the genes chosen from the group consisting of:
the gap gene which codes for glyceraldehyde 3-phosphate dehydrogenase (Eikmanns (1992), Journal of Bacteriology 174: 6076-6086),
the tpi gene which codes for triose phosphate isomerase (Eikmanns (1992), Journal of Bacteriology 174: 6076-6086),
the pgk gene which codes for 3-phosphoglycerate kinase (Eikmanns (1992), Journal of Bacteriology 174: 6076-6086),
the zwf gene which codes for glucose 6-phosphate dehydrogenase (JP-A-09224661, WO 01/70995),
the pyc gene which codes for pyruvate carboxylase (EP-A-1083225),
the lysC gene which codes for a feed-back resistant aspartate kinase (Accession No.P26512; EP-B-0387527; EP-A-0699759; WO 00/63388),
the hom gene which codes for homoserine dehydrogenase (EP-A 0131171),
the metA gene which codes for homoserine O-acetyltransferase (ACCESSION Number AF052652),
the metb gene which codes for cystathionine gamma-synthase (ACCESSION Number AF126953),
the aecD gene which codes for cystathionine gamma-lyase (ACCESSION Number M89931),
the metY gene which codes for O-acetylhomoserine sulfhydrylase (DE 10043334, DSM 13556),
the glyA gene which codes for serine hydroxymethyltransferase (JP-A-08107788),
   may be enhanced and, in particular, over-expressed.

Furthermore, it may be advantageous for the production of amino acids, in addition to the attenuation of the metD gene, at the same time for one or more of the genes chosen from the group consisting of:
the pck gene which codes for phosphoenol pyruvate carboxykinase (EP-A-1094111),
the pgi gene which codes for glucose 6-phosphate isomerase (EP-A-1087015, WO 01/07626),
the poxB gene which codes for pyruvate oxidase (EP-A-1096013),
the thrB gene which codes for homoserine kinase (ACCESSION Number P8210),
the thrC gene which codes for threonine synthase (ACCESSION Number P23669),
the metK gene which codes for methionine adenosyltransferase (ACCESSION Number AJ290443)
the ddh gene which codes for meso-diaminopimelate D-dehydrogenase (ACCESSION Number Y00151),
   to be attenuated and, in particular, for the expression thereof to be reduced.

In addition to the attenuation of the metD gene it may furthermore be advantageous for the production of amino acids to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The invention also provides the microorganisms prepared according to the invention, and these can be cultured continuously or discontinuously in the batch process (batch culture) or in the fed batch (feed process) or repeated fed batch process (repetitive feed process) for the purpose of production of L-amino acids. A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

The substances:
(a) sugars and carbohydrates, such as e.g. glucose, sucrose, lactose, fructose, maltose, molasses, starch and cellulose,
(b) oils and fats, such as, soya oil, sunflower oil, groundnut oil and coconut fat,
(c) fatty acids, such as palmitic acid, stearic acid and linoleic acid,
(d) alcohols, such as glycerol and ethanol, and
(e) organic acids, such as acetic acid,
   may be used individually, or as a mixture, as the source of carbon.

The substances:
(a) Organic nitrogen-containing compounds, such as peptones, yeast extract, meat extract, malt extract, corn steep liquor, soya bean flour and urea, or
(b) inorganic compounds, such as ammonium sulfate, ammonium chloride, ammonium phosphate, ammonium carbonate and ammonium nitrate, can be used be used individually, or as a mixture, as the source of nitrogen.

Phosphoric acid, potassium dihydrogen phosphate or dipotassium hydrogen phosphate or the corresponding sodium-containing salts can be used as the source of phosphorus.

The culture medium must furthermore comprise salts of metals, such as magnesium sulfate or iron sulfate, which are necessary for growth.

Essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture.

Antifoams, such as, for example, fatty acid polyglycol esters, can be employed to control the development of foam. Suitable substances having a selective action, such as, for example, antibiotics, can be added to the medium to maintain the stability of plasmids. To maintain aerobic conditions, oxygen or oxygen-containing gas mixtures, such as air, are introduced into the culture. The temperature of the culture is usually 20° C. to 45° C., and preferably 25° C. to 40° C. Culturing is continued until a maximum of the desired product has formed. This target is usually reached within 10 hours to 160 hours.

The fermentation broth prepared in this manner, in particular containing L-methionine, is then further processed. Depending on requirements, all or some of the biomass can be removed from the fermentation broth by separation methods. Examples of such separation methods are centrifugation, filtration, decanting or a combination thereof. Alternatively, the biomass can be left completely in the fermentation broth. This broth is can optionally be thickened or concentrated by known methods. Examples of such thickening or concentrating methods include conventional methods such as evaporation, reverse osmosis, or by nanofiltration. Examples of instruments that can be used in evaporation processes include methods a rotary evaporator, thin film evaporator, and falling film evaporator. This thickened or concentrated fermentation broth can then be worked up. Examples of methods used to work up the thickened or concentrated fermentation broth include freeze drying, spray drying, spray granulation or by other processes. Optionally, the fermentation broth can be worked up to yield a preferably free-flowing, finely divided powder.

The free-flowing, finely divided powder can be converted by suitable compacting or granulating processes. Preferably, the powder can be converted into a coarse-grained, readily free-flowing, storable and largely dust-free product. During granulation or compaction, it is advantageous to employ conventional organic or inorganic auxiliary substances or carriers. Examples of such organic or inorganic auxiliary substances or carriers include starch, gelatin, cellulose derivatives or similar substances. Further, these substances can be used as binders, gelling agents or thickeners in foodstuffs or feedstuffs processing. Further examples of these substances include silicas, silicates or stearates.

“Free-flowing” is understood as meaning powders which flow unimpeded out of the vessel with the opening of 5 mm (millimeters) of a series of glass outflow vessels with outflow openings of various sizes (Klein, Seifen, Öle, Fette, Wachse 94, 12 (1968)).

As described here, “finely divided” means a powder with a predominant content, i.e. >50%, with a particle size of from 20 to 200 μm diameter. The ranges for the particle size include all specific values and subranges therebetween, such as 30, 40, 50, 60, 70, 80, 90, 100, 110, 120, 130, 140, 150, 160, 170, 180, and 190 μm diameter. “Coarse-grained” means products with a predominant content, i.e. >50%, with a particle size of 200 to 2000 μm diameter. The ranges for the particle size include all specific values and subranges therebetween, such as 200, 300, 400, 500, 600, 700, 800, 900, 1000, 1100, 1200, 1300, 1400, 1500, 1600, 1700, 1800, and 1900 μm diameter. In this context, “dust-free” means that the product contains only small contents, i.e. <5%, with particle sizes of less than 20 μm diameter. The particle size determination can be carried out with methods of laser diffraction spectrometry. The corresponding methods are described in the textbook on “Teilchengröβenmessung in der Laborpraxis” by R. H. Müller and R. Schuhmann, Wissenschaftiliche Verlagsgesellschaft Stuttgart (1996) or in the textbook “Introduction to Particle Technology” by M. Rhodes, Verlag Wiley & Sons (1998).

“Storable” in the context of this invention means a product which can be stored for up to 120 days, preferably up to 52 weeks, particularly preferably 60 months, without a substantial loss, i.e. <5%, of methionine.

Alternatively, however, the product can be absorbed onto an organic or inorganic carrier substance which is known and conventional in feedstuffs processing. Examples of such organic or inorganic carrier substances include silicas, silicates, grits, brans, meals, starches, sugars or others. Further, the product simultaneously or subsequently mixed and/or stabilized with conventional thickeners and/or binders. Examples of uses and processes in this context are described in the literature (Die Mühle+Mischfuttertechnik 132 (1995) 49, page 817).

Finally, the product can be brought into a state in which it is stable to digestion by animal stomachs, in particular the stomach of ruminants, by coating processes, i.e. coating. Examples of such conventional coating processes include those that use film-forming agents. Examples of film-forming agents include metal carbonates, silicas, silicates, alginates, stearates, starches, gums and cellulose ethers, which are described in DE-C-4100920.

If the biomass is separated off during the process, further inorganic solids which can be optionally added during the fermentation can be optionally removed. In addition, the animal feedstuffs additive according to the invention can optionally comprise a predominant proportion, i.e. >50%, of further substances. Examples of such further substances include organic substances which can be optionally formed and/or added and are optionally present in solution in the fermentation broth, because they have optionally not been separated off by suitable processes.

In one aspect of the invention, the biomass can be separated off to the extent of up to 70%, preferably up to 80%, preferably up to 90%, preferably up to 95%, and particularly preferably up to 100%. In another aspect of the invention, up to 20% of the biomass, preferably up to 15%, preferably up to 10%, preferably up to 5%, particularly preferably no biomass is separated off.

Examples of the above-mentioned organic substances include organic by-products. Organic by-products can be optionally produced, in addition to the L-methionine, and can be optionally discharged by the microorganisms employed in the fermentation. Examples of organic by-products include L-amino acids chosen from the group consisting of L-valine, L-threonine, L-alanine or L-tryptophan. Further examples of organic by-products include vitamins chosen from the group consisting of vitamin B1 (thiamine), vitamin B2 (riboflavin), vitamin B5 (pantothenic acid), vitamin B6 (pyridoxine), vitamin B12 (cyanocobalamin), nicotinic acid/nicotinamide and vitamin E (tocopherol). Even further examples of organic by-products include organic acids. Examples of organic acids are those that contain one to three carboxyl groups. Examples of organic acids containing one to three carboxyl groups include acetic acid, lactic acid, citric acid, malic acid and/or fumaric acid. Finally, Examples of organic by-products include sugars. Examples of sugars include such trehalose. These compounds are optionally desired if they improve the nutritional value of the product.

Organic substances, including L-methionine and/or D-methionine and/or the racemic mixture D,L-methionine, can optionally be added during a suitable process step. Organic substances can be in many forms. Examples of such forms include concentrate and/or pure substance in solid and/or liquid form. These organic substances mentioned can optionally be added individually or as mixtures to the resulting or concentrated fermentation broth, or also optionally during the drying or granulation process. It is likewise possible to optionally add an organic substance or a mixture of several organic substances to the fermentation broth and a optionally add further organic substance or a further mixture of several organic substances during a later process step. Examples of such as later step can include a granulation step.

The product described above is suitable as a feedstuffs additive, i.e. feed additive, for animal nutrition.

The L-methionine content of the animal feedstuffs additive is conventionally 1 wt. % to 80 wt. %, preferably 2 wt. % to 80 wt. %, particularly preferably 4 wt. % to 80 wt. %, and very particularly preferably 8 wt. % to 80 wt. %, based on the dry weight of the animal feedstuffs additive. Contents of 1 wt. % to 60 wt. %, 2 wt. % to 60 wt. %, 4 wt. % to 60 wt. %, 6 wt. % to 60 wt. %, 1 wt. % to 40 wt. %, 2 wt. % to 40 wt. % or 4 wt. % to 40 wt. % are likewise possible. The ranges for content of the animal feedstuffs additive include all specific values and subranges therebetween, such 2, 4, 6, 8, 10, 15, 20, 25, 30, 35, 40, 45, 50, 55, 60, 65, 70, and 75 wt. %. The water content of the feedstuffs additive is conventionally up to 5 wt. %, preferably up to 4 wt. %, and particularly preferably less than 2 wt. %.

An animal feedstuffs additive according to the present invention can comprise 1 wt. % to 80 wt. % L-methionine, D-methionine, D,L-methionine, or a mixture thereof with 1 to 40 wt. % L-lysine, D-lysine or D,L-lysine, based on the dry weight of the animal feedstuffs additive. The ranges for content of L-methionine, D-methionine, D,L-methionine, or a mixture thereof with L-lysine, D-lysine or D,L-lysine in the animal feedstuffs additive include all specific values and subranges therebetween, such 2, 4, 6, 8, 10, 15, 20, 25, 30, 35, 40, 45, 50, 55, 60, 65, 70, and 75 wt. %. The ranges for content of L-lysine, D-lysine or D,L-lysine in the mixture with L-methionine, D-methionine, D,L-methionine in the animal feedstuffs additive include all specific values and subranges therebetween, such 2, 4, 6, 8, 10, 15, 20, 25, 30, and 35 wt. %.

The invention accordingly also provides a process for the preparation of an L-methionine-containing animal feedstuffs additive from fermentation broths, which comprises the steps:
h) culturing an L-methionine-producing microorganism in a fermentation medium;
i) concentrating the L-methionine-containing fermentation broth;
j) removing an amount of from 0 to 100 wt. % of the biomass formed during the fermentation; and
k) drying of the fermentation broth obtained according to a) and/or b) to obtain the animal feedstuffs additive in the desired powder or granule form.

The concentrating step b) of the above-mentioned process includes the removal of substances from the L-methionine-containing fermentation broth. Examples of such substances that can be removed include water.

If desired, one or more of the following steps can furthermore be carried out in the process according to the invention:
l) adding at least one organic substances, including L-methionine and/or D-methionine and/or the racemic mixture D,L-methionine, to the products obtained according to a), b) and/or c);
m) adding auxiliary substances chosen from the group consisting of silicas, silicates, stearates, grits and bran to the substances obtained according to a) to d) for stabilization and to increase the storability; or
n) converting the substances obtained according to a) to e) into a form stable to the animal stomach, in particular rumen, by coating with film-forming agents.

The analysis of L-methionine can be carried out by ion exchange chromatography with subsequent ninhydrin derivation, as described by Spackman et al. (Analytical Chemistry, 30, (1958), 1190).

Methods for the determination of L-amino acids are known from the prior art. The analysis can thus be carried out, for example, as described by Spackman et al. (Analytical Chemistry, 30, (1958), 1190) by anion exchange chromatography with subsequent ninhydrin derivation, or it can be carried out by reversed phase HPLC, for example as described by Lindroth et al. (Analytical Chemistry (1979) 51: 1167-1174).

The process according to the invention is used for fermentative preparation of amino acids.

The isolation of plasmid DNA from Escherichia coli and all techniques of restriction, Klenow and alkaline phosphatase treatment were carried out by the method of Sambrook et al. (Molecular Cloning. A Laboratory Manual, 1989, Cold Spring Harbor Laboratory Press, Cold Spring Harbor, N.Y., USA). Methods for transformation of Escherichia coli are also described in this handbook.

The composition of the usual nutrient media, such as LB or TY medium, can also be found in the handbook by Sambrook et al.

The present invention is explained in more detail with the aid of the following embodiment examples.

### EXAMPLE

The abbreviations and designations used have the following meaning:

| | |
|---|---|
| oriV | ColE1-similar origin from pMB1 |
| sacB | sacB gene coding for the protein levansucrase |
| KmR | Kanamycin resistance |
| HindIII | Cleavage site of the restriction enzyme HindIII |
| SwaI | Cleavage site of the restriction enzyme SwaI |
| RP4mob | RP4 mobilization site |
| metD del | Cloned deletion derivative for metD |

### Example 1

Preparation of a Genomic Cosmid Gene Library from C. glutamicum ATCC 13032

Chromosomal DNA from C. glutamicum ATCC 13032 is isolated as described by Tauch et al. (1995, Plasmid 33: 168-179) and partly cleaved with the restriction enzyme Sau3AI (Amersham Pharmacia, Freiburg, Germany, Product Description Sau3AI, Code no. 27-0913-O₂). The DNA fragments are dephosphorylated with shrimp alkaline phosphatase (Roche Molecular Biochemicals, Mannheim, Germany, Product Description SAP, Code no. 1758250). The DNA of the cosmid vector SuperCos1 (Wahl et al. (1987), Proceedings of the National Academy of Sciences, USA 84: 2160-2164), obtained from Stratagene (La Jolla, USA, Product Description SuperCos1 Cosmid Vector Kit, Code no.251301) is cleaved with the restriction enzyme XbaI (Amersham Pharmacia, Freiburg, Germany, Product Description XbaI, Code no. 27-0948-O₂) and likewise dephosphorylated with shrimp alkaline phosphatase.

The cosmid DNA is then cleaved with the restriction enzyme BamHI (Amersham Pharmacia, Freiburg, Germany, Product Description BamHI, Code no. 27-0868-04). The cosmid DNA treated in this manner is mixed with the treated ATCC 13032 DNA and the batch is treated with T4 DNA ligase (Amersham Pharmacia, Freiburg, Germany, Product Description T4-DNA-Ligase, Code no.27-0870-04). The ligation mixture is then packed in phages with the aid of Gigapack II XL Packing Extract (Stratagene, La Jolla, USA, Product Description Gigapack II XL Packing Extract, Code no. 200217).

For infection of the E. coli strain NM554 (Raleigh et al. 1988, Nucleic Acid Res. 16: 1563-1575) the cells are taken up in 10 mM MgSO₄ and mixed with an aliquot of the phage suspension. The infection and titering of the cosmid library are carried out as described by Sambrook et al. (1989, Molecular Cloning: A Laboratory Manual, Cold Spring Harbor), the cells being plated out on LB agar (Lennox, 1955, Virology, 1:190)+100 mg/] ampicillin. After incubation overnight at 37° C., recombinant individual clones are selected.

### Example 2

Isolation and Sequencing of the metD Gene

The cosmid DNA of an individual colony is isolated with the Qiaprep Spin Miniprep Kit (Product No. 27106, Qiagen, Hilden, Germany) in accordance with the manufacturer's instructions and partly cleaved with the restriction enzyme Sau3A1 (Amersham Pharmacia, Freiburg, Germany, Product Description Sau3AI, Product No. 27-0913-O₂). The DNA fragments are dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 bp are isolated with the QiaExII Gel Extraction Kit (Product No. 20021, Qiagen, Hilden, Germany).

The DNA of the sequencing vector pZero-1, obtained from Invitrogen (Groningen, Holland, Product Description Zero Background Cloning Kit, Product No. K2500-01) is cleaved with the restriction enzyme BamHI (Amersham Pharmacia, Freiburg, Germany, Product Description BamHI, Product No. 27-0868-04). The ligation of the cosmid fragments in the sequencing vector pZero-1 is carried out as described by Sambrook et al. (1989, Molecular Cloning: A Laboratory Manual, Cold Spring Harbor), the DNA mixture being incubated overnight with T4 ligase (Pharmacia Biotech, Freiburg, Germany). This ligation mixture is then electroporated (Tauch et al. 1994, FEMS Microbiol. Letters, 123: 343-7) into the E. coli strain DH5αmcr (Grant, 1990, Proceedings of the National Academy of Sciences, U.S.A., 87: 4645-4649) and plated out on LB agar (Lennox, 1955, Virology, 1:190) with 50 mg/l zeocin.

The plasmid preparation of the recombinant clones is carried out with a Biorobot 9600 (Product No. 900200, Qiagen, Hilden, Germany). The sequencing is carried out by the dideoxy chain-stopping method of Sanger et al. (1977, Proceedings of the National Academies of Sciences, U.S.A., 74: 5463-5467) with modifications according to Zimmermann et al. (1990, Nucleic Acids Research, 18: 1067). The “RR drhodamin Terminator Cycle Sequencing Kit” from PE Applied Biosystems (Product No. 403044, Weiterstadt, Germany) was used. The separation by gel electrophoresis and analysis of the sequencing reaction are carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained are then processed using the Staden program package (1986, Nucleic Acids Research, 14: 217-231) version 97-0. The individual sequences of the pZerol derivatives are assembled to a continuous contig. The computer-assisted coding region analysis is prepared with the XNIP program (Staden, 1986, Nucleic Acids Research 14: 217-231). Further analyses are carried out with the “BLAST search programs” (Altschul et al., 1997, Nucleic Acids Research, 25: 3389-3402) against the non-redundant databank of the “National Center for Biotechnology Information” (NCBI, Bethesda, Md., USA).

The resulting nucleotide sequence is shown in SEQ ID No. 1. Analysis of the nucleotide sequence shows an open reading frame of 711 bp, which is called the metD gene. The metD gene codes for a polypeptide of 236 amino acids.

The DNA sections lying upstream and downstream of SEQ ID No. 1 were identified in the same way, these sections being shown in SEQ ID No. 3 and SEQ ID No. 4. The metD gene region extended by SEQ ID No. 3 and SEQ ID No. 4 is shown in SEQ ID No. 5.

### Example 3

Incorporation of a Deletion into the metD Gene

For this, chromosomal DNA is isolated from the strain ATCC13032 by the method of Tauch et al. (Plasmid 33: 168-179 (1995)). On the basis of the sequence of the metD gene known for C. glutamicum from example 2, the oligonucleotides described below are chosen for generation of the metD deletion allele by means of the polymerase chain reaction (PCR) by the gene Soeing method (Horton, Molecular Biotechnology 3: 93-98 (1995)).

Primer metD -DelA (see also SEQ ID No. 6):
5′-GAT CTA AAG CTT-GCC TCT CCA ATC TCC ACT GA-3′
Primer metD -DelB (see also SEQ ID No. 7):
5′-ATT GAG TAG TCC GCA GGT GG-ATT TAA AT-AAT CCA CAG GCA AGT CTA GC-3′
Primer metD -DelC (see also SEQ ID No. 8):
5′-GCT AGA CTT GCC TGT GGA TT-ATT TAA AT-CCA CCT GCG GAC TAC TCA AT-3′
Primer metD -DelD (see also SEQ ID No. 9):
5′-GAT CTA AAG CTT-GAT GTC CAT GTA CCG CAG C-3′

The primers shown are synthesized by MWG Biotech (Ebersberg, Germany) and the PCR reaction is carried out using Pfu polymerase (Stratagene, Product. No. 600135, La Jolla, USA) and a PTC 100 Thermocycler (MJ Research Inc., Waltham, USA).

The primers metD-DelA and metD-DelD contain in each case an inserted cleavage site for the restriction enzyme HindIII, and the primers melD-DelB and metD-DelC an inserted cleavage site for the restriction enzyme SwaI, which are marked by underlining in the nucleotide sequence shown above.

The primer melD-DelB is composed of two regions of the nucleotide sequence, one of which bonds in the coding sequence of metD to the nucleotides 707 to 688, and the other bonds to the “upstream” region in front of the start codon of metD. Both regions are divided by the inserted SwaI restriction enzyme site. The Primer metD-DelC is reverse complementary to the Primer metD-DelB.

With the aid of the polymerase chain reaction the primers metD-DelA and metD-DelB enable the amplification of a 573-bp DNA fragment and the Primers metD-DelC and metD-DelD enable the amplification of a 651 bp DNA fragment. The amplificates are examined by subsequent agarose-gel electrophoresis in an 0,8% agarose-gel, isolated from the agarose-gel with the High Pure PCR Product Purification Kit (Product No. 1732676, Roche Diagnostics GmbH, Mannheim, Deutschland), and used together as a DNA template in another PCR reaction using the primers metD-DelA and metD-DelD. This results in the production of the melD deletion derivative, 1177 bp in size (see also SEQ ID No. 10).

The amplified product is subsequently examined in a 0.8% agarose-gel.

### Example 4

4.1 Construction of the Exchange Vector pK18mobsacBmetD del

The metD deletion derivative obtained in example 3 is cleaved with the restriction endonuclease HindIII, after examination in a 0,8% agarose-gel isolated from the agarose gel with the High Pure PCR Product purification Kit (Product No. 1732676, Roche Diagnostics GmbH, Mannheim, Deutschland) and used for ligation with the mobilizable cloning vector pK18mobsacB described by Schäfer et al., Gene, 14, 69-73 (1994). The vector pK18mobsacB was cleaved beforehand with the restriction enzyme HindIII and subsequently dephosphorylated with shrimp alkaline phosphatase (Roche Diagnostics GmbH, Mannheim, Germany, Product Description SAP, Product No. 1758250). The prepared vector is then mixed with the metD deletion derivative and treated with T4 DNA ligase (Amersham-Pharmacia, Freiburg, Germany).

The E. coli strain DH5αmcr (Grant, 1990, Proceedings of the National Academy of Sciences U.S.A., 87: 4645-4649) is then electroporated with the ligation batch (Hanahan, In. DNA cloning. A practical approach. Vol.1. ILR-Press, Cold Spring Harbor, N.Y., 1989). Selection of plasmid-carrying cells is made by plating out the transformation batch on LB agar (Sambrook et al., Molecular Cloning: A Laboratory Manual, 2^{nd} Ed., Cold Spring Harbor, N.Y., 1989), which has been supplemented with 25 mg/l kanamycin.

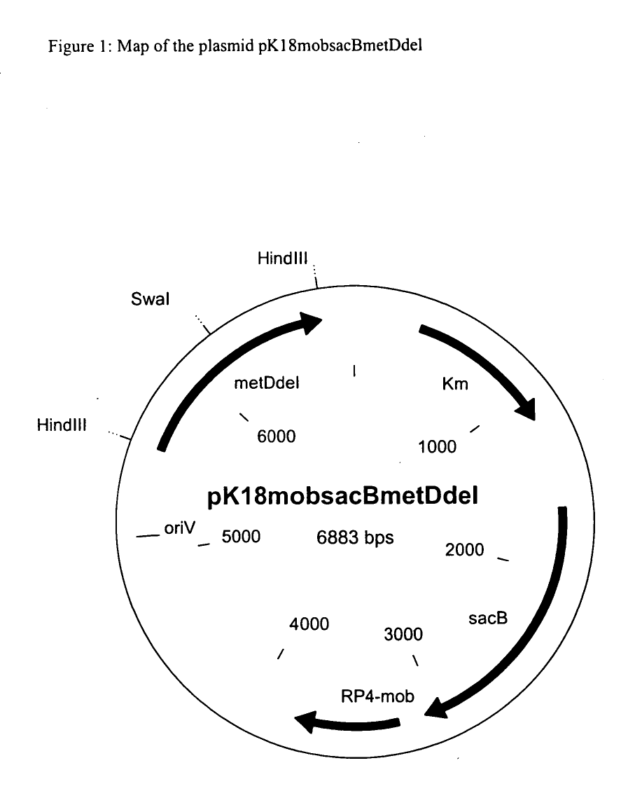

Plasmid DNA is isolated from a transformant with the aid of the QIAprep Spin Miniprep Kit from Qiagen and verified by cutting with the restriction enzymes HindIII and Swal. The plasmid is called pK18mobsacBmetD del and is shown in FIG. 1. The strain is called E. coli DH5αmcr/pK18mobsacBmetD del.

4.2 Deletion Mutagenesis of the metD Gene in the C. glutamicum Strain ATCC13032

The vector pK18mobsacBmetD del mentioned in example 4.1 is electroporated by the electroporation method of Tauch et al., (1989 FEMS Microbiology Letters 123: 343-347) in the strain C. glutamicum ATCC13032. The vector cannot replicate independently in ATCC13032 and is retained in the cell only if it has integrated into the chromosome. Selection of clones with integrated pK18mobsacmetD del is carried out by plating out the electroporation batch on LB agar (Sambrook et al., Molecular Cloning: A Laboratory Manual, 2^{nd} Ed., Cold Spring Harbor, N.Y., 1989), which has been supplemented with 15 mg/l kanamycin. Clones which had grown on are plated out on LB agar plates with 25 mg/l kanamycin and incubated for 16 hours at 33° C.

To achieve excision of the plasmid together with the complete chromosomal copy of the metD gene, the clones are incubated unselectively overnight in LB medium and then cultured on LB agar with 10% sucrose. The plasmid pK18mobsacB contains a copy of the sacB gene, which converts sucrose into levan sucrose, which is toxic to C. glutamicum. Only those clones in which the integrated pK18mobsacBmetD del has been excised again therefore grow on LB agar with sucrose. In the excision, together with the plasmid either the complete chromosomal copy of the metD gene can be excised, or the metD deletion derivative.

To demonstrate that the metD gene is deleted in the chromosome, the plasmid pK18mobsacBmetD del is marked by the method of “The DIG System Users Guide for Filter Hybridization” of Boehringer Mannheim GmbH (Mannheim, Germany, 1993) using the DIG hybridization kit from Boehringer. Chromosomal DNA of a potential deletion mutant is isolated by the method of Eikmanns et al. (Microbiology 140: 1817-1828 (1994)) and in each case cleaved with the restriction enzymes HindIII and SwaI in separate batches. The fragments formed are separated by agarose gel electrophoresis and hybridized at 68° C. with the Dig hybridization kit from Boehringer. With the aid of the fragments formed, it can be shown that the strain ATCC13032 has lost its copy of the metD gene, and instead carries the deleted allele.

The strain is called C. glutamicum ATCC13032ΔmetD.

### Example 5

Production of Methionine

The C. glutamicum strain ATCC13032ΔmetD obtained in example 4 is cultured in a nutrient medium suitable for the production of methionine and the methionine content in the culture supernatant is determined.

For this, the strain is first incubated on a brain-heart agar plate for 24 hours at 33° C. Starting from this agar plate culture, a preculture is seeded (10 ml medium in a 100 ml conical flask). The medium MM is used as the medium for the preculture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.01 | mg/l |
| Vitamin B12 (sterile-filtered) | 0.02 | mg/l |
| Thiamine * HCl (sterile-filtered) | 0.2 | mg/l |
| CaCO₃ | 25 | g/l |

The CSL, MOPS and the salt solution are brought to pH 7 with aqueous ammonia and autoclaved. The sterile substrate and vitamin solutions are then added, as well as the CaCO₃ autoclaved in the dry state.

The preculture is incubated for 16 hours at 33° C. at 240 rpm on a shaking machine. A main culture is seeded from this preculture such that the initial OD (660 nm) of the main culture was 0.1. Medium MM is also used for the main culture.

Culturing is carried out in a 10 ml volume in a 100 ml conical flask with baffles. Culturing is carried out at 33° C. and 80% atmospheric humidity.

After 72 hours, the OD is determined at a measurement wavelength of 660 nm with a Biomek 1000 (Beckmann Instruments GmbH, Munich). The amount of methionine formed is determined with an amino acid analyzer from Eppendorf-BioTronik (Hamburg, Germany) by ion exchange chromatography and post-column derivation with ninhydrin detection.

The result of the experiment is shown in Table 1.

TABLE 1

| | OD | Methionine |
|---|---|---|
| Strain | (660 nm) | mg/l |
| ATCC13032 | 12.2 | 3 |
| ATCC13032ΔmetD | 14.8 | 20 |

Numerous modifications and variations on the present invention are possible in light of the above teachings. It is, therefore, to be understood that within the scope of the accompanying claims, the invention may be practiced otherwise than as specifically described herein.

The present application claims priority to German Application No. DE 10126164.0, filed May 30, 2001. The entire content of this application is incorporated herein by reference.

1-30. (canceled).

31. Escherichia coli DH5αmcr/pK18mobsacBmetD del.

32. A process for producing an L-amino acids comprising:
culturing a bacterial cell in a medium suitable for producing an L-amino acids, wherein the metD gene in said bacterial cell has been eliminated or inactivated, or wherein expression of the metD gene has been attenuated.

33. The process of claim 32, wherein said bacterial cell is a Coryneform bacterium.

34. The process of claim 33, wherein the bacterial cell is selected from the group consisting of Corynebacterium glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

35. The process of claim 32, wherein the metD gene comprises the polynucleotide sequence of SEQ ID NO. 1, or is at least 90% identical to SEQ ID NO: 1 and encodes a polypeptide which regulates transcription.

36. The process of claim 32, wherein the L-amino acid is L-methionine.

37. The process of claim 32, wherein the bacteria further comprises at least one gene whose expression is enhanced selected from the group consisting of the gap, tpi, pgk, zwf pyc, lysC, hom, metA, metB, aecD, metY, and glyA.

38. The process of claim 32, wherein the bacteria further comprises at least one gene whose expression is attenuated selected from the group consisting of the pck, pgi, poxB, thrB, thrC, metK, and ddh.

39. The process of claim 32, wherein the bacterial cell is Corynebacterium glutamicum strain ATCC13032deltametD.

40-41. (canceled)

42. A process for the preparation of an animal food additive, comprising:
a) culturing at least one L-methionine-producing microorganism in a fermentation medium;
b) removing water from the fermentation medium;
c) removing from 0 to 100 wt. % of the biomass from the fermentation medium formed during the culturing; and
d) drying the fermentation medium.

43. The process according to claim 42, wherein the at least one microorganism comprises genes of the biosynthesis pathway of L-methionine are that are enhanced.

44. The process according to claim 42, wherein the at least one microorganism comprises genes of the biosynthesis pathway of L-methionine are that are attenuated.

45. The process according to claim 42, wherein the at least one microorganism comprises a polynucleotide which encodes a metD gene, wherein the metD gene is attenuated.

46. The process according to claim 42, wherein the at least one microorganism is Corynebacterium glutamicum.

47. The process according to claim 42, wherein the at least one microorganism is Corynebacterium glutamicum strain ATCC13032deltametD.

48. The process according to claim 42, further comprising at least one of the following steps:
e) adding at least one organic substance selected from the group consisting of L-methionine and D-methionine to the fermentation medium;
f) adding at least one auxiliary substance selected from the group consisting of silicas, silicates, stearates, grits, and bran to the fermentation medium; or
g) converting the fermentation medium obtained from at least one step selected from the group consisting a), b), c), d), e), and f) into an animal food additive.

49. The process according to claim 48, wherein the converting is performed by coating the fermentation medium with at least one film-forming agent.

50. An animal food additive made by the process according to claim 49, wherein the animal food additive is stable in the stomach of an animal.

51. An animal food additive made by the process according to claim 49, wherein the animal food additive is stable in the rumen of an animal.

52. An animal food additive made by the process according to claim 49, wherein the at least one film-forming agent is selected from the group consisting of metal carbonates, silicas, silicates, alginates, stearates, starches, gums, and cellulose ethers.


SEQUENCE LISTING (11 sequences)

SEQ ID NO: 1 - DNA, 1322 bp, Corynebacterium  glutamicum
  CDS (314)..(1021)
agattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaacgtggctgctagcgcttcaggcaagagtaaaacaagtgccggggcaaaccgtcgtcgcaatcgaccaagcccccgacagcgtctcctcgatagcgcaaccaaccttttcaccacagaaggtattcgcgtcatcggtattgatcgtatcctccgtgaagctgacgtggcgaaggcgagcctctattcccttttcggatcgaaggacgccttggttattgcatacctggagaacctcgatcagctgtggcgtgaagcgtggcgtgagcgcaccgtcggtatgaaggatccggaagataaaatcatcgcgttctttgatcagtgcattgaggaagaaccagaaaaagatttccgcggctcgcactttcagaatgcggctagtgagtaccctcgccccgaaactgatagcgaaaagggcattgttgcagcagtgttagagcaccgcgagtggtgtcataagactctgactgatttgctcactgagaagaacggctacccaggcaccacccaggcgaatcagctgttggtgttccttgatggtggacttgctggatctcgattggtccacaacatcagtcctcttgagacggctcgcgatttggctcggcagttgttgtcggctccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttc

SEQ ID NO: 2 - protein, 236 aa, Corynebacterium  glutamicum
LSILVLDSSSYLETDQPERTWPNVAASASGKSKTSAGANRRRNRPSPRQRLLDSATNLFTTEGIRVIGIDRILREADVAKASLYSLFGSKDALVIAYLENLDQLWREAWRERTVGMKDPEDKIIAFFDQCIEEEPEKDFRGSHFQNAASEYPRPETDSEKGIVAAVLEHREWCHKTLTDLLTEKNGYPGTTQANQLLVFLDGGLAGSRLVHNISPLETARDLARQLLSAPPADYSI

SEQ ID NO: 3 - DNA, 239 bp, Artificial Sequence
  Synthetic DNA
gcctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctcca

SEQ ID NO: 4 - DNA, 289 bp, Artificial Sequence
  Synthetic DNA
tttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacccaatagagtgcgattgctgtggggaatggtcctgtgaggccaagggacagtgggaagatcggcgcgaggatcgacatcacgatcatgaacttcagcatgccgttagagaatccggatgcgtaatcgttggtttggaagctgcggtacatggacatc

SEQ ID NO: 5 - DNA, 1850 bp, Corynebacterium  glutamicum
  CDS (553)..(1260)
gcctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaacgtggctgctagcgcttcaggcaagagtaaaacaagtgccggggcaaaccgtcgtcgcaatcgaccaagcccccgacagcgtctcctcgatagcgcaaccaaccttttcaccacagaaggtattcgcgtcatcggtattgatcgtatcctccgtgaagctgacgtggcgaaggcgagcctctattcccttttcggatcgaaggacgccttggttattgcatacctggagaacctcgatcagctgtggcgtgaagcgtggcgtgagcgcaccgtcggtatgaaggatccggaagataaaatcatcgcgttctttgatcagtgcattgaggaagaaccagaaaaagatttccgcggctcgcactttcagaatgcggctagtgagtaccctcgccccgaaactgatagcgaaaagggcattgttgcagcagtgttagagcaccgcgagtggtgtcataagactctgactgatttgctcactgagaagaacggctacccaggcaccacccaggcgaatcagctgttggtgttccttgatggtggacttgctggatctcgattggtccacaacatcagtcctcttgagacggctcgcgatttggctcggcagttgttgtcggctccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacccaatagagtgcgattgctgtggggaatggtcctgtgaggccaagggacagtgggaagatcggcgcgaggatcgacatcacgatcatgaacttcagcatgccgttagagaatccggatgcgtaatcgttggtttggaagctgcggtacatggacatc

SEQ ID NO: 6 - protein, 236 aa, Corynebacterium  glutamicum; identical to SEQ ID NO: 2

SEQ ID NO: 7 - DNA, 32 bp, Artificial Sequence
  Synthetic DNA
gatctaaagcttgcctctccaatctccactga

SEQ ID NO: 8 - DNA, 48 bp, Artificial Sequence
  Synthetic DNA
attgagtagtccgcaggtggatttaaataatccacaggcaagtctagc

SEQ ID NO: 9 - DNA, 48 bp, Artificial Sequence
  Synthetic DNA
gctagacttgcctgtggattatttaaatccacctgcggactactcaat

SEQ ID NO: 10 - DNA, 31 bp, Artificial Sequence
  Synthetic DNA
gatctaaagcttgatgtccatgtaccgcagc

SEQ ID NO: 11 - DNA, 1177 bp, Artificial Sequence
  Synthetic DNA
gatctaaagcttgcctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattatttaaatccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacccaatagagtgcgattgctgtggggaatggtcctgtgaggccaagggacagtgggaagatcggcgcgaggatcgacatcacgatcatgaacttcagcatgccgttagagaatccggatgcgtaatcgttggtttggaagctgcggtacatggacatcaagctttagatc